(19) United States
(12) Patent Application Publication
(10) Pub. No.: US 2006/0036250 A1
(43) Pub. Date: Feb. 16, 2006

(54) Antero-lateral plating systems for spinal stabilization

(76) Inventors: Eric C. Lange, Collierville, TN (US); Anthony J. Melkent, Memphis, TN (US); Kent Anderson, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/037,023

(22) Filed: Jan. 18, 2005

Related U.S. Application Data
(60) Provisional application No. 60/600,893, filed on Aug. 12, 2004.

(51) Int. Cl. A61F 2/30

(52) U.S. Cl. 606/69

(57) ABSTRACT

A plating system for stabilization of a bony segment includes a plate engageable to at least first and second bony elements. For spinal stabilization, the plate is attached to the antero-lateral portions of at least first and second vertebrae and is structured to facilitate engagement of the plate to the vertebrae from an approach extending in the anterior-posterior directions.

### CROSS-REFERENCE TO RELATED APPLICATION

This application claims the benefit of the filing date of U.S. Provisional Application No. 60/600,893 filed on Aug. 12, 2004, which is incorporated herein by reference in its entirety.

### BACKGROUND

Various types of plating devices and systems have been used to stabilize portions of bones including the spine. Spinal stabilization techniques have employed plating on the posterior, anterior, lateral, postero-lateral and antero-lateral portions of a spinal column segment. Such plating systems can provide fixation of a spinal column segment for the repair of injured or diseased vertebrae, intervertebral discs, and other elements of the spinal column. There remains a need for spinal plating systems that address antero-lateral fixation of the spinal column.

### BRIEF DESCRIPTION OF THE DRAWINGS

FIG. 1 is a perspective view of one embodiment antero-lateral plate.

FIG. 2 is an end view of the plate of FIG. 1.

FIG. 3 is a bottom perspective view of the plate of FIG. 1.

FIG. 4 is an end view of the plate of FIG. 1 with bone engaging fasteners positioned therethrough.

FIG. 5 is a perspective view of a spinal column segment with the plate of FIG. 1 positioned thereon.

FIG. 6 is a perspective view of another embodiment antero-lateral plate.

FIG. 7 is an end perspective view of the plate of FIG. 6.

FIG. 8 is an end view of the plate of FIG. 6 with bone engaging fasteners positioned therethrough.

FIG. 9 is a perspective view of the spinal column segment with the plate of FIG. 6 positioned thereon.

FIG. 10 is an elevational view of the spinal column segment with the plate of FIG. 1 engaged thereto along one antero-lateral side of the spinal column segment.

FIG. 11 is an elevational view of the spinal column segment with plates of FIG. 6 positioned along each antero-lateral side of the spinal column segment.

FIG. 12 is a perspective view looking toward the upper surface of another embodiment antero-lateral plate.

FIG. 13 is a perspective view looking toward the lower surface of the plate of FIG. 12.

FIG. 14 is a medial-lateral section view through the plate of FIG. 12.

FIG. 15 is an elevation view of the plate of FIG. 12 positioned along a vertebral level of the spinal column.

FIG. 16 is an end elevation view of the plate of FIG. 12 with bone engaging fasteners positioned therethrough.

FIG. 17 is an end elevation view showing the plate engaged antero-laterally to a vertebral body.

FIG. 18 is a plan view showing plates engaged antero-laterally along multiple vertebral levels.

FIG. 19 is a view looking along the spinal midline showing an anterior surgical approach for engaging an antero-lateral plate to the spinal column.

FIG. 20 is an end view of two plates positioned along a vertebral body and an intradiscal coupling mechanism interconnecting the plates.

FIG. 21 is a view looking along the spinal midline showing an instrument and technique for engaging another antero-lateral plate embodiment to the spinal column.

FIG. 22 is an elevation view in partial-section showing a portion of the instrument of FIG. 21.

### SUMMARY

According to one aspect, a method for securing a plate to at least two vertebrae includes accessing the vertebrae from a direct anterior approach; positioning a plate antero-laterally along the at least two vertebrae, and engaging bone fasteners to the vertebrae through plate along an approach generally parallel to the sagittal plane.

According to another aspect, a method for securing a plate to at least two vertebrae includes accessing the vertebrae from a direct anterior approach; positioning a fusion construct through a portal formed by the approach in a disc space between the at least two vertebrae, positioning a plate through the portal and antero-laterally along the at least two vertebrae, and engaging bone fasteners to the vertebrae through plate along an anterior-posterior approach extending anteriorly through the portal.

According to another aspect, a plating system for stabilization of at least first and second vertebrae includes a plate having at least a first hole therethrough between an upper surface and a lower surface of the plate to receive a bone fastener for passage into the first vertebra and at least a second hole therethrough between an upper surface and a lower surface of said plate to receive a bone fastener for passage into the second vertebra. The lower surface of the plate is adapted for placement along an antero-lateral portion of the first and second vertebrae, and the first and second holes extend along first and second axes, respectively. The first and second hole axes are oriented non-orthogonally to the lower surface of the plate.

According to another aspect, a plating system for stabilization of at least first and second vertebrae includes a plate having at least a first hole therethrough between an upper surface and a lower surface of the plate to receive a bone fastener for passage into the first vertebra and at least a second hole therethrough between an upper surface and a lower surface of said plate to receive a bone fastener for passage into the second vertebra. The lower surface of the plate is adapted for placement along an antero-lateral portion of the first and second vertebrae, and the first and second holes extend along first and second axes, respectively. The first and second axes are oriented non-orthogonally to the lower surface of the plate. The upper and lower surfaces extend between a lateral edge and a medial edge of the plate, the lateral edge defining a thickness that is greater than a thickness of the plate at the medial edge.

According to another aspect, a plating system for stabilization of first and second vertebrae includes a plate having at least a first hole therethrough between an upper surface and a lower surface of the plate to receive a bone fastener for passage into the first vertebra and at least a second hole therethrough between an upper surface and a lower surface of said plate to receive a bone fastener for passage into the second vertebra. The lower surface of the plate is adapted for placement along an antero-lateral portion of the first and second vertebrae, and the first and second holes extend along first and second axes, respectively. The first and second axes are oriented non-orthogonally to the lower surface of the plate, and the upper surface includes a stair-stepped configuration.

According to another aspect, an antero-lateral plate includes an upper surface and a lower surface positionable antero-laterally along at least two vertebrae. The upper surface includes a stair-stepped configuration having a first portion adjacent a medial edge of the plate and a second portion adjacent a lateral edge of the plate. The first and second portions are parallel to one another, and each includes at least one hole extending orthogonally therethrough for receiving a bone fastener to secure the plate to the at least two vertebrae.

According to another aspect, a plating system includes first and second plates positionable antero-laterally along vertebrae and on opposite sides of the sagittal plane. An intradiscal, length adjustable coupling mechanism interconnects the first and second plates.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and any such further applications of the principles of the invention as illustrated therein are contemplated as would normally occur to one skilled in the art to which the invention relates.

Antero-lateral plates are provided for attachment to a bony segment of the human body, such as two or more vertebrae of the spinal column. In some applications, placement of the plate in antero-lateral position can minimize intrusion or contact of the plate with the great vessels extending anteriorly along the spinal column. The plate is attachable to the antero-lateral or oblique aspect of the two or more vertebrae in an anterior approach to the spine. When in the antero-lateral position the plates are configured to receive bone fasteners from an anterior-posterior trajectory through the anterior approach to minimize intrusion into tissue lateral of the anterior approach. The plate can be employed for antero-lateral spinal stabilization in conjunction with anterior fusion procedures through the same anterior approach created for placement of the fusion construct in a disc space between vertebrae.

It is contemplated that the plates can be attached to any one or combination of the cervical, thoracic, lumbar and sacral regions of the spinal column. The plates can be employed uni-laterally, i.e. a single plate attached to the vertebrae along one side of the midline of the spinal column. The plates can also be employed bi-laterally, i.e. two plates attached to the vertebra on opposite sides of the midline of the spinal column. In either uni-lateral or bi-lateral employment of the plates, multiple plates may be employed to stabilize the same vertebral levels or multiple levels of the spinal column. The plates can also be modular for attachment to one another to form a plate assembly extending along multiple vertebral levels.

The plate can be fixed to each vertebra by at least one bone engaging fastener adjacent each end of the plate. In one specific embodiment, the plate includes a first portion positionable along an upper vertebra, a second portion positionable along a lower vertebra, and a middle portion therebetween extending along the spinal disc space between the adjacent vertebrae. The first portion includes a pair of holes for receiving bone engaging fasteners to engage the plate to the upper vertebra, and the second portion includes a pair of holes for receiving bone engaging fasteners to engage the plate to the lower vertebra. In a further embodiment, the plates includes a third portion having at least one hole for receiving a bone engaging fastener to engage the plate to a third vertebra.

The plate may be provided with one or more retaining members that are engagable to or attached to the plate and which resist the bone fasteners from backing out of the plate holes in situ. Examples of such retaining devices are provided in U.S. Pat. Nos. 6,152,927; 6,533,786; 5,364,399, and U.S. patent application Ser. No. 10/219,516; each of which is incorporated herein by reference in its entirety. Other embodiment retaining members are also contemplated, including snap rings positioned in, about or adjacent each of or multiple ones of the plate holes. The snap rings can allow passage, therethrough or thereagainst, of the bone engaging fastener into the plate hole and into the vertebra. A contact surface, a mating receptacle, or other structure formed by the bone engaging fastener aligns with and contacts or receives the snap ring as it returns toward its pre-insertion configuration. Interference between the snap ring and the bone engaging fastener prevents or resists back-out of the bone engaging fastener relative to the plate.

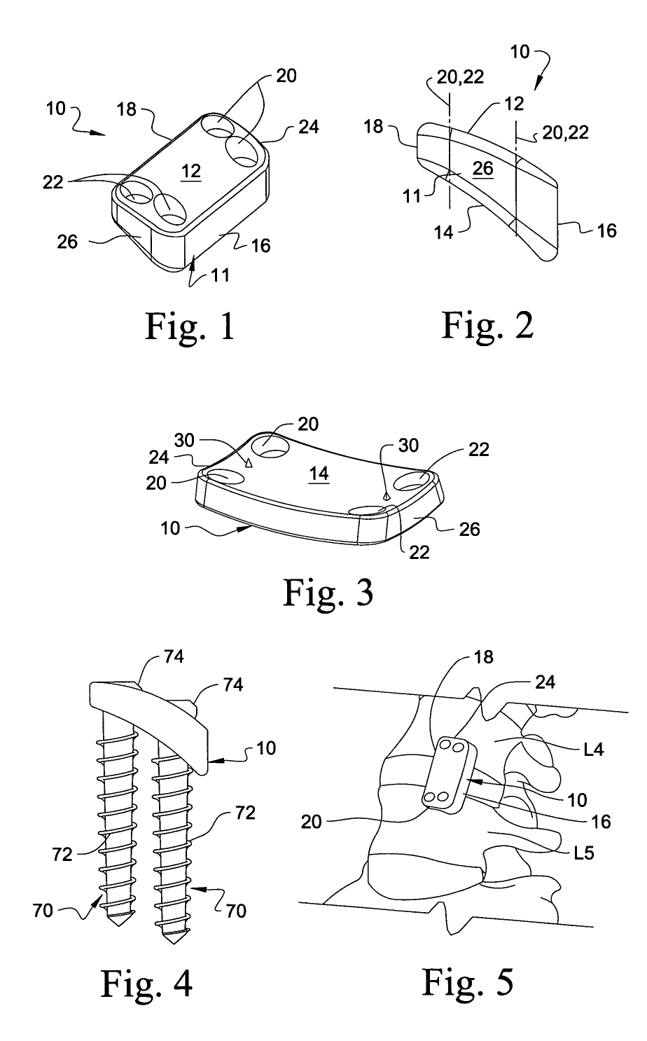

Referring to FIGS. 1-5, there is shown a plate 10 attachable to first and second vertebrae of a spinal column segment. Plate 10 includes a body 11 having an upper surface 12 and an opposite lower surface 14. Body 11 extends between a lateral side 16 and a medial side 18, and also between a cephalad end 24 and a caudal end 26. A pair of first holes 20 are provided adjacent cephalad end 24 and a pair of second holes 22 are provided adjacent caudal end 26.

Holes 20, 22 extend between and open at upper and lower surfaces 12, 14, and are sized to received a bone fastener therethrough, as shown in FIG. 4. Other embodiments contemplate that only one hole is provided adjacent one or both of the ends 24, 26. Still other embodiments contemplate more than two holes at one or both of the ends 24, 26. Further embodiments contemplate body 11 is sized to extend along three or more vertebrae, and that one or more holes are provided through body 11 at each vertebral level between cephalad end 24 and caudal end 26.

As shown in FIG. 3, one embodiment of plate 10 includes lower surface 14 having spikes 30 projecting therefrom adjacent each of cephalad end 24 and caudal end 26. Spikes 30 can be positioned into respective ones of first and second vertebrae to temporarily secure plate 10 thereto prior to insertion of the bone engaging fasteners. Other embodiments contemplate a plate with more than two spikes 30, one spike 30, or no spikes 30. Still other embodiments contemplate a plate having spikes with no holes for receiving bone engaging fasteners. The spike can engage the plate to the vertebrae, and/or the plate can be coupled to one or both of an intradiscal and extradiscal stabilization construct to secure it to the spinal column segment.

Lower surface 14 can include a concave curvature between lateral side 16 and medial side 18 adapted to conform to the antero-lateral curvature of the vertebral bodies against which plate 10 is to be positioned. Upper surface 12 includes a convex curvature between lateral side 16 and medial side 18. In the illustrated embodiment, the thickness of body 11 is greater at lateral side 16 than at medial side 18. As shown in FIG. 5, this allows lateral side 16 of plate 11 to extend laterally around the vertebral bodies along lower surface 14, while minimizing the intrusion of upper surface 12 of body 11 into the surrounding tissue anteriorly of body 11 and laterally of lateral side 16.

As shown in FIG. 2, holes 20, 22 extend along axes that are non-orthogonal to lower surface 14. In this configuration, the axes of holes 22, 24 are oriented so that their axes extend in the anterior-posterior direction which is generally parallel to the sagittal plane when plate 10 is position in an antero-lateral location along the vertebrae, as shown in FIG. 5. Bone engaging fasteners 70 are shown in FIG. 4, and are positionable through holes 20, 22 to engage plate 10 to the respective vertebrae of the spinal column segment. In the illustrated embodiment, bone engaging fasteners 70 include a threaded shaft 72 projecting below lower surface 14 for engaging with the underlying bony structure. Bone engaging fasteners 70 further include an enlarged head 74 residing in respective ones of the holes 20, 22 adjacent upper surface 12. Enlarged head 74 contacts body 11 and secures plate 10 against the bony structure when head 74 is positioned against body 11.

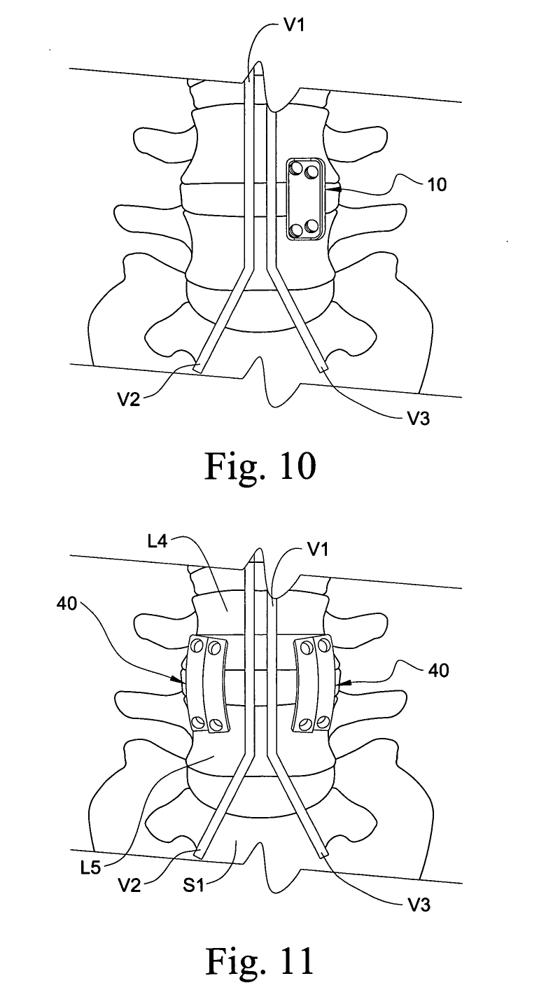

As shown in FIG. 5 and also at least in FIGS. 10, 17 and 18 for the other plate embodiments, one embodiment of the implanted orientation for plate 10 contemplates antero-lateral positioning along the L4 and L5 vertebrae. Cephalad end 24 is located along vertebra L4, and caudal end 26 is located along vertebra L5. Medial side 18 is oriented medially or toward the spinal mid-line, and lateral side 16 is oriented laterally or away from the spinal mid-line. Plate 10 is positioned, relative to the patient, on the left hand side of the spinal mid-line. Other embodiments contemplate a mirror image of plate 10 for placement on the right hand side of the spinal midline.

In its implanted orientation, plate 10 is located laterally of the great vessels V1 extending along the spinal midline. In the illustrated embodiment, placement of the plate on the L4-L5 vertebrae locates the plate cephaladly of the bifurcation V2, V3 of the great vessels V1. The intrusiveness of the procedure is minimized since plate 10 avoids contact with the great vessels along the anterior side of the spinal column. Plate 10 can be positioned along the spinal column through the same approach taken for access to the disc space between the L4 and L5 vertebrae for placement of a fusion construct. The invasiveness of the procedure is minimized since external stabilization can be provided without a posterior intrusion for placement of a plate or rod construct. Also, alignment of the axes of holes 20, 22 in the portal formed by the anterior approach minimizes intrusion laterally into tissue along the approach during placement of the bone fasteners through the plate holes.

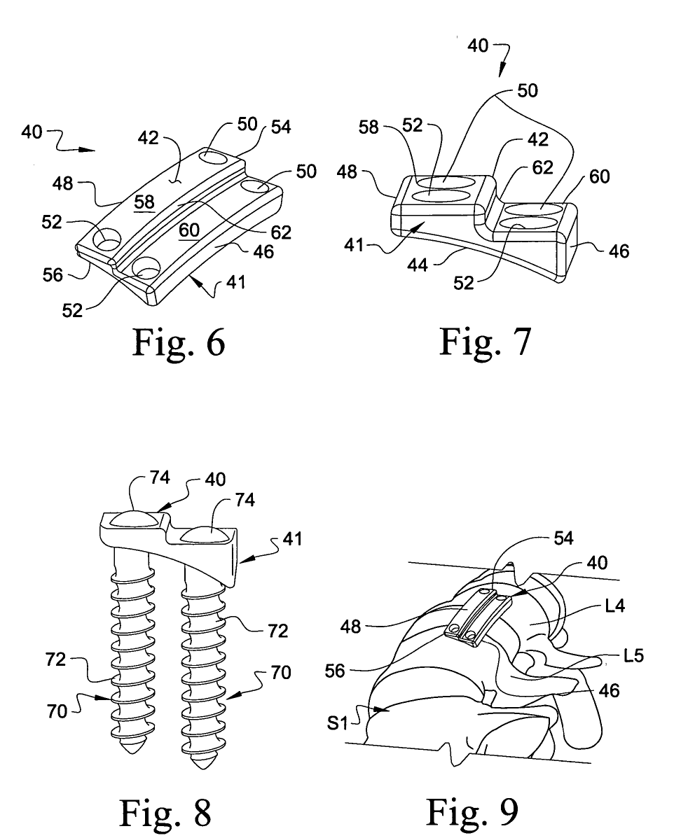

Another embodiment plate 40 is shown in FIGS. 6-9. Plate 40 attachable to first and second vertebrae of a spinal column segment, such as vertebrae L4 and L5 shown in FIG. 9. Plate 40 includes a body 41 having an upper surface 42 and an opposite lower surface 44. Body 41 extends between a lateral side 46 and a medial side 48, and also between a cephalad end 54 and a caudal end 56. A pair of first holes 50 are provided adjacent cephalad end 54 and a pair of second holes 52 are provided adjacent caudal end 56. Holes 50, 52 extend between and open at upper and lower surfaces 42, 44, and are sized to received a bone engaging fastener therethrough, as shown in FIG. 8.

Other embodiments contemplate that one hole is provided adjacent one or both of the ends 54, 56. Still other embodiments contemplate more than two holes at one or both of the ends 54, 56. Further embodiments contemplate body 41 is sized to extend along three or more vertebrae, and that one or more holes are provided through body 41 at each vertebral level between cephalad end 54 and caudal end 56.

Lower surface 44 can include a concave curvature between lateral side 46 and medial side 48 adapted to conform to the antero-lateral curvature of the vertebral bodies against which plate 40 is to be positioned. Upper surface 42 includes a first portion 58 adjacent medial side 48 and a second portion 60 adjacent lateral side 46. A riser portion 62 extends between first and second portions 58, 60. Upper surface 42 thus forms a stair-stepped configuration extending between the medial and lateral sides 48, 46. In the illustrated embodiment, the thickness of body 41 is greater at lateral side 46 than at medial side 48. As shown in FIG. 9, this allows lateral side 46 of body 41 to extend laterally around the vertebral bodies along lower surface 44, while minimizing the intrusion of upper surface 42 of body 41 into the surrounding tissue located anteriorly of body 41 and laterally of lateral side 46.

As shown in FIG. 7, holes 50, 52 extend along axes that are non-orthogonal to lower surface 44, but orthogonally oriented to the portion of upper surface 42 extending along respective ones of the first and second portions 58, 60. In this configuration, holes 50, 52 are oriented so that their axes extend in the anterior-posterior directions and generally parallel with the sagittal plane when plate 10 is position in an antero-lateral location along the vertebrae, as shown in FIG. 9. Bone engaging fasteners 70 are shown in FIG. 8, and are positionable through holes 50, 52 to engage plate 40 to the respective vertebrae of the spinal column segment. Upper surface portions 58, 60 extend generally parallel to one another, and can act as a guide for placement of bone engaging fasteners 70 through the holes 50, 52.

As shown in FIG. 9 and also in FIG. 11, one embodiment of the implanted orientation for plate 40 contemplates antero-lateral positioning along the L4 and L5 vertebrae. Cephalad end 54 is located along vertebra L4, and caudal end 56 is located along vertebra L5. Medial side 48 is oriented medially or toward the spinal mid-line, and lateral side 46 is oriented laterally or away from the spinal mid-line. In FIG. 9, one plate 40 is positioned, relative to the patient, on the left hand side of the spinal mid-line. Other embodiments contemplate a mirror image of plate 40 for placement on the right hand side of the spinal midline, such as shown in FIG. 11, to provide bi-lateral extradsical stabilization of the vertebral level.

Similar to plate 10, in its implanted orientation plate 40 is located laterally of the great vessels V1 extending along the spinal midline. In the illustrated embodiment, placement of the plate on the L4-L5 vertebrae locates the plate cephaladly of the bifurcation V2, V3 of the great vessels V1. The intrusiveness of the procedure is minimized since plate 40 avoids contact with the great vessels along the anterior side of the spinal column. Plate 10 can be positioned along the spinal column through the same portal forming the anterior approach taken for access to the disc space between the L4 and L5 vertebrae for placement of a fusion construct. The invasiveness of the procedure is minimized since external stabilization can be provided without posterior intrusion for placement of a plate or rod construct. Also, alignment of the axes of holes 50, 52 generally parallel with the sagittal plane in the portal of the anterior approach minimizes intrusion laterally into tissue along the approach for placement of the bone fasteners through the plate holes.

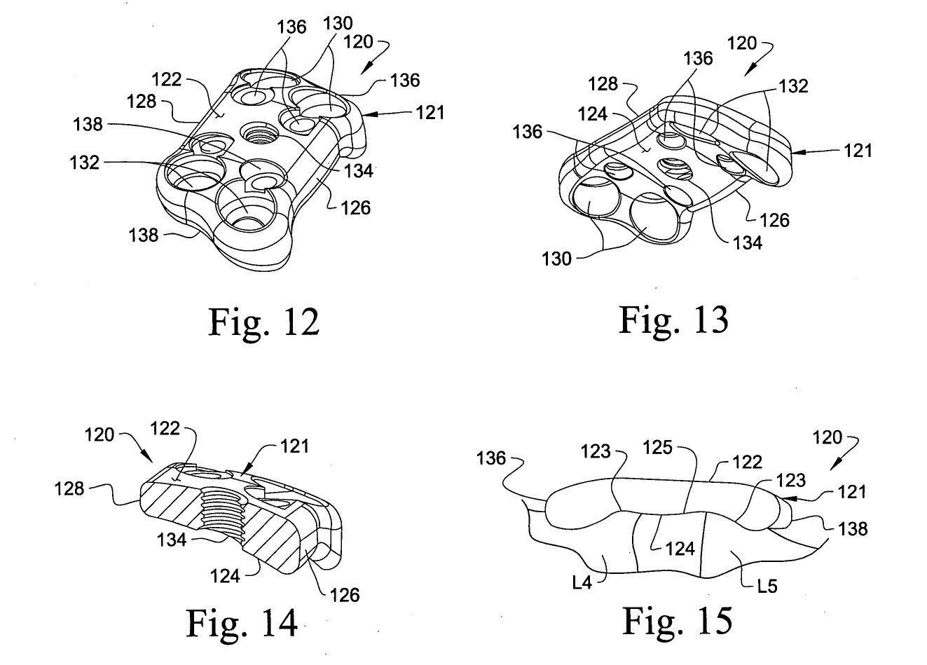

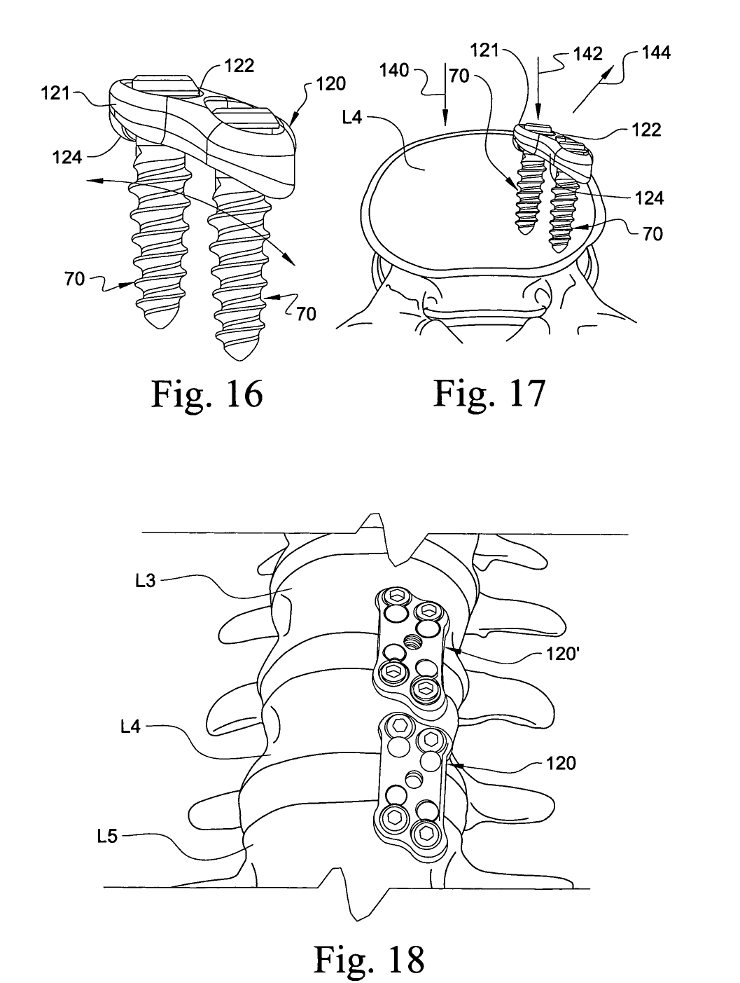

Referring now to FIGS. 12-13, there is shown another embodiment plate 120 for antero-lateral stabilization of a spinal column segment. Plate 120 includes a body 121 having an upper surface 122 and an opposite lower surface 124. Body 121 extends between a lateral side 126 and a medial side 128, and also between a cephalad end 136 and a caudal end 138. A pair of first holes 130 are provided adjacent cephalad end 136 and a pair of second holes 132 are provided adjacent caudal end 138. Holes 130, 132 extend between and open at upper and lower surfaces 122, 124, and are sized to received a bone engaging fastener therethrough, as shown in FIGS. 16-17. The corners of plate 120 about holes 130, 132 and the transitions between adjacent plate surfaces can be smooth and rounded to eliminate sharp or abrupt corners or transitions that might impinge on adjacent tissue and anatomical structures.

Plate 120 is further shown with first bores 136 adjacent respective ones of the first holes 130 and second bores 138 adjacent respective ones of the second holes 132. First and second bores 136, 138 can receive retaining mechanisms to secure and/or prevent the bone engaging fasteners from backing out of the plate holes. Such retaining mechanisms may include set screws, snap rings, screw and washer combinations, or any other retaining mechanism embodiment. There is further provided a central bore 134 that can be engaged with an inserter, drill guide or other instrument to facilitate placement and securement of plate 120 along the spinal column. In still a further embodiment, a fastener can be engaged to central bore 134 to secure a retaining member on upper surface 122 of plate 120. In still another embodiment, central bore 134 can include one or more bores for receiving one or more fasteners to engage an interbody device or a vertebral body through plate 120. Other embodiments of plate 120 contemplate that any or all of the bores 134, 136, 138 are not provided.

As shown in FIGS. 14, 16 and 17, lower surface 124 can include a concave curvature between lateral side 126 and medial side 128 adapted to conform to the antero-lateral curvature of the vertebral bodies against which plate 120 is to be positioned. Upper surface 122 includes a convex curvature between lateral side 126 and medial side 128. In the illustrated embodiment, the thickness of body 121 is greater at lateral side 126 than at medial side 128. As discussed above with respect to other plate embodiments, this allows lateral side 126 of plate 120 to extend laterally around the vertebral bodies along lower surface 124, while minimizing the intrusion of upper surface 122 of body 121 into the surrounding tissue toward medial side 128.

As shown in FIG. 15, lower surface 124 of plate 120 can also be curved to conform to the vertebral surface profile in the cephalad and caudal directions. For example, body 121 may include a convexly curved discal portion 125 positionable extradiscally along the intervertebral disc between vertebrae, and concavely curved pockets 123 at each end of portion 125 to receive the cortical rim of the respective adjacent vertebra. The portions of lower surface 122 extending along cephalad and caudal ends 136, 138 are each angled to slope away from upper surface 122 and in the same direction relative to the adjacent discal portion 125 to conform to the outer surface profile of the adjacent vertebral bodies. The cephalad-caudal curvature in combination with the medial-lateral curvature of body 121 forms a low profile footprint projecting outwardly from the vertebral bodies.

As shown in FIGS. 16-17, one embodiment of plate 120 includes lower surface 124 curved to conform to the antero-lateral profile of the vertebrae in the medial-lateral direction. As discussed above with respect to the other plate embodiments, such curvature facilitates placement of plate 120 along an anterior-posterior placement axis 142 in an anterior approach 140 to the spinal column segment. The axes of holes 130, 132 also extend in a direction parallel to anterior placement axis 142, which is also parallel to the sagittal plane. The axes of holes 130, 132 are obliquely oriented relative to lower surface 124 of plate 120. In the operative position, the axes of holes 130, 132 are also obliquely oriented relative to a normal axis 144, which is also normal to the vertebral surfaces along which lower surface 124 is placed.

Referring now to FIG. 18, there is shown a multi-level stabilization placement of plates 120 along multiple levels of the spinal column. The cephalad ends of each of the plates 120, 120′ include staggered profiles so that the medial side of each plate extends more cephaladly than the lateral side. The caudal ends of each of the plates 120, 120′ include a staggered profile so that the lateral side extends more caudally than the medial side. The cephalad and caudal ends of the plates 120, 120′ also include concavely curved or recessed end wall portions between the plate holes to nestingly receive a respective node or projecting portion of the other plate member, guiding the positioning of the plates relative to one another and allowing placement of the plates closely to one another.

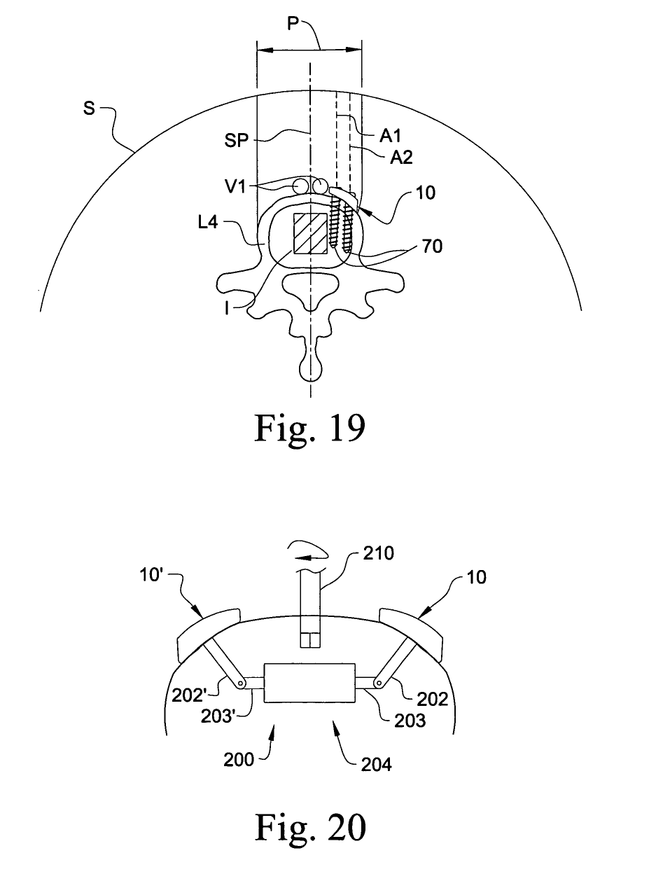

Referring now to FIG. 19, a procedure with plate 10 will be describe, it being understood that the procedure also has application with the other plate embodiments discussed herein. An anterior incision is made in skin S of the patient and an access portal P is formed to access one or more vertebral levels including vertebra L4, it being understood that access to other vertebral levels not including vertebra L4 is also contemplated. Access portal P provides a direct anterior approach to the spinal column that is generally centered about the sagittal plane SP. Great vessels V1 are manipulated with a retractor or other instrument for accessing a spinal disc space for preparation of the disc space and vertebra to receive a fusion construct I. Fusion construct I may include any one or more of a threaded fusion cage, a push-in cage, a bone implant, a spacer, bone graft; and bone growth material and therapeutic substances. Fusion construct I can be symmetrical or asymmetrical relative to sagittal plane SP. Placement of fusion construct I can be guided or facilitated with guide sleeves, retractors, ramps, inserters, or any other device or instrument for placement of the same. It is further contemplated that fusion constructs can be employed at multiple levels along the spinal column.

With fusion construct I in the disc space between vertebrae, plate 10 can be positioned along the antero-lateral aspect of the vertebrae between which the fusion construct is positioned. Lower surface 14 is shaped to conform to this antero-lateral profile, while upper surface 12 is oriented toward and accessible in portal P. In some procedures, the soft tissues along portal P can be shifted laterally to provide additional space for placement of and access to plate 10.

Bone fasteners 70 are then positioned through plate holes 20, 22 to secure plate 10 to the vertebrae. Bone fasteners 70 can be positioned along approach axes A1 and A2 extending from holes 20, 22. Approach axes A1, A2 extend generally parallel to sagittal plane SP, thus allowing the bone fasteners to be engaged to the vertebrae without additional retraction or displacement of tissue to accommodate placement of bone fasteners 70. A standard straight driver instrument can be employed through portal P to engage the bone fasteners 70 to the vertebrae. If bi-lateral stabilization is desired, a second plate can be engaged antero-laterally to the vertebrae on the opposite side of sagittal plane SP. The plate embodiments discussed herein provide for antero-lateral stabilization with plate and fastener placement through the same direct anterior approach employed for an anterior interbody fusion procedure.

The plate embodiments discussed herein can include cephalad and caudal ends that are angled toward one another toward the medial side of the plate to limit the medial dimension of the plate and also to allow placement of the plate holes closer to the central axis of the plate, minimizing the width of the plate. The corners of the plate body transitioning between the sides and ends of the plate body can be rounded to eliminate sharp or abrupt edges that could pinch, cut or wear against tissue. The surfaces of the plate body transitioning between the upper and lower surfaces can also be smooth and rounded to eliminate sharp or abrupt edges that could pinch, cut or wear against tissue.

It is contemplated that the overall configuration of the plates can be standardized and provided in a range of sizes. The external dimensions of the plate can also vary depending on the patient anatomy determined according to standardized measurements or pre-operative modeling of the region to be stabilized. The plates can have a shape suited for antero-lateral attachment to vertebrae of a spinal column segment in the cervical, thoracic, lumbar and sacral regions.

The plate holes and fasteners can be configured such that the fasteners have a fixed angle orientation relative to the plate or variable angle orientations. One or more of the holes can be circular, or elongated to allow translation of the fastener along the hole. The holes can include a recessed surface extending thereabout that allows the head of the bone fastener to be recessed into the plate, minimizing extension of the fastener from the plate into the tissue adjacent the plate. One or more retaining members may be employed with the plate to prevent the fasteners from backing out of the plate holes, to engage the fasteners in the plate holes, and/or to fix the fasteners in the plate holes.

Referring now to FIG. 20, there is shown another embodiment procedure where a first plate 10 is positioned antero-laterally along one side of the spinal column and a second plate 10′ is positioned antero-laterally along another side of the spinal column. The bi-lateral, antero-laterally positioned plates are connected to one another through an intradiscal coupling mechanism 204. Coupling mechanism 204 can be an interbody device, connector bar, or fusion construct in the disc space that extends between the plates.

To accommodate variability of the anatomy of the spinal column segment and the spacing between plates, in one embodiment the coupling mechanism includes a connector bar that is length adjustable. In a further embodiment, the connector bar includes flexible or hinged connections to the plates to allow adjustment in the relative orientation between the connector bar and the plates. In still another embodiment, the coupling mechanism is a turnbuckle, center screw, or a slider-lever, for example.

In one specific embodiment, the connector bar includes a spinning connector piece that interconnects links 203, 203′. The spinning piece can be mounted to and freely rotatable to one of the links 203, 203′, and threadingly engaged to the other of the links 203, 203′. Rotation of the spinning piece shortens or lengthens coupling mechanism 204 between plates 10, 10′.

Still further it is contemplated that plates 10, 10′ may include connecting arms 202, 202′ pivotally and/or slidably connected to coupling mechanism 204. The arms 202, 202′ pivot relative to coupling mechanism 204 as it is shortened or lengthened. As coupling mechanism 204 is shortened, plates 10, 10′ are drawn toward and into firm engagement with the adjacent antero-lateral vertebral surfaces. The pivotal connections can be provided by a ball joint, universal joint, pinned joint, or other suitable connector. The connector can be lockable to secure connecting arms, 202, 202′, links 203, 203′ and/or coupling mechanism 204 in a desired relative orientation with plates 10, 10′.

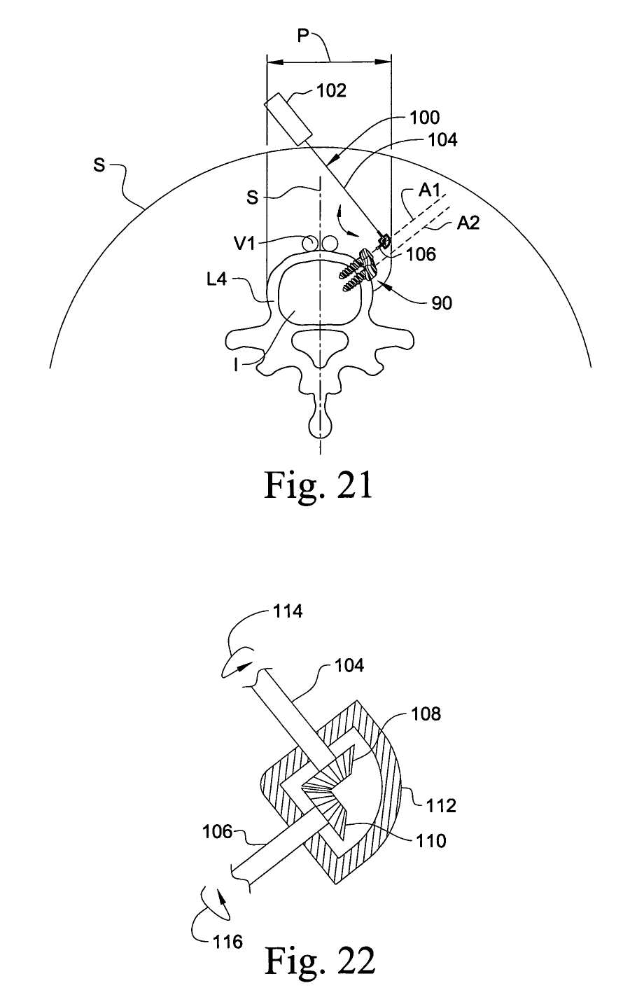

Referring now to FIGS. 21 and 22, there is shown another embodiment procedure for placement and engagement of a plate 90 antero-laterally along two or more vertebrae in conjunction with direct anterior placement of a fusion construct in one or more disc spaces between vertebrae. Plate 90 includes fasteners 70 extending therethrough along axes A1, A2 that are orthogonal to the lower surface of plate 90 and obliquely oriented to the sagittal plane. Accordingly, when plate 90 is positioned antero-laterally as shown in FIG. 13, the hole axes A1, A2 project outside the portal P forming the direct anterior approach to the vertebrae.

An instrument 100 is provided that is structured to engage bone fasteners 70 in the holes of plate 100 while instrument 100 is positioned in portal P. Instrument 100 includes a handle 102, a first shaft portion 104, and a second shaft portion 106 angularly and rotatably coupled to first shaft portion 104. As shown in FIG. 22, one embodiment connection mechanism between shaft portions 104, 106 include a beveled gear 108 at a distal end of first shaft portion 104, and second shaft portion 106 includes a beveled gear 110 at a proximal end thereof. Gears 108, 110 can be received in housing 112 to prevent pinching or wearing of the gears against tissue or other anatomical structures during rotation.

Gears 108, 110 interact with one another so that rotation of first shaft portion 104 in the direction indicated by arrow 112 effects rotation of second shaft portion 106 in the direction indicated by arrow 116. The distal end of second shaft portion 106 can be mounted to the bone fastener extending through a hole in plate 90 and threadingly advances the bone fastener into the plate holes as it is rotated. It is further contemplated that the distal shaft portion 106 can be provided with a modular configuration that accepts attachments for various procedures that may be performed with instrument 100. Such modular tips may include drill, tap, awl, or screwdriver attachments, for example.

While the invention has been illustrated and described in detail in the drawings and foregoing description, the same is to be considered as illustrative and not restrictive in character, it being understood that only preferred embodiments have been shown and described and that all changes and modifications that come within the spirit of the invention are desired to be protected.

What is claimed is:

1. A method for securing a plate to at least two vertebrae of a patient, comprising:
accessing the vertebrae from a direct anterior approach;
positioning a plate antero-laterally along the at least two vertebrae on a first side of a sagittal plane of the patient; and
positioning bone fasteners into the at least two vertebrae along an approach generally parallel to the sagittal plane to secure the plate to the at least two vertebrae.

2. The method of claim 1, wherein positioning bone fasteners includes threadingly engaging a pair of bone fasteners to each of the at least two vertebrae along the approach.

3. The method of claim 1, wherein positioning bone fasteners includes positioning each bone fastener into the vertebrae in a direction generally parallel to the sagittal plane.

4. The method of claim 1, wherein positioning the plate includes embedding spikes extending from a lower surface of the plate into the at least two vertebrae.

5. The method of claim 1, wherein the plate includes a first thickness along a laterally positioned side thereof that is greater than a second thickness along a medially positioned side thereof.

6. The method of claim 5, wherein the plate includes a lower surface concavely curved between the medial and lateral sides.

7. The method of claim 6, wherein the plate includes an upper surface convexly curved between the medial and lateral sides.

8. The method of claim 6, wherein the plate includes an upper surface having a first portion lying in a first plane, a second portion lying in a second plane parallel to the first plane, and a riser portion therebetween.

9. The method of claim 8, wherein the plate includes:
a first hole in the first portion extending between the upper and lower surfaces orthogonally oriented to the first plane; and
a second hole in the second portion extending between the upper and lower surfaces and orthogonally oriented to the second plane.

10. The method of claim 1, further comprising:
positioning a second plate antero-laterally along the at least two vertebrae on a second side of the sagittal plane; and
positioning bone fasteners into the at least two vertebrae along a second approach generally parallel to the sagittal plane to secure the second plate to the at least two vertebrae.

11. The method of claim 10, further comprising engaging the first and second plates to one another with a length adjustable coupling mechanism extending through a disc space between the at least two vertebrae.

12. The method of claim 1, further comprising:
positioning a second plate antero-laterally on the first side of the sagittal plane along at least one of the two vertebrae and along at least one other vertebra; and
positioning bone fasteners along an approach generally parallel to the sagittal plane to secure the second plate to the respective vertebrae.

13. A method for securing a plate to at least two vertebrae of a patient, comprising: accessing the vertebrae from a direct anterior approach;
positioning a fusion construct through a portal formed by the direct anterior approach and into a disc space between the at least two vertebrae;
positioning a plate through the portal and antero-laterally along the at least two vertebrae, wherein in the antero-lateral position the plate is completely offset to a side of the sagittal plane; and
engaging bone fasteners to the vertebrae in an anterior-posterior direction to secure the plate to the vertebrae.

14. The method of claim 13, wherein engaging bone fasteners includes engaging a pair of bone fasteners to each of the at least two vertebrae along the anterior-posterior approach.

15. The method of claim 13, wherein engaging bone fasteners includes positioning the bone fasteners through the portal.

16. The method of claim 13, wherein positioning the plate includes embedding spikes extending from a lower surface of the plate into the at least two vertebrae.

17. The method of claim 13, wherein the plate includes a first thickness along a laterally positioned side thereof that is greater than a second thickness along a medially positioned side thereof.

18. The method of claim 17, wherein the plate includes a lower surface concavely curved between the medial and lateral sides.

19. A plating system for stabilization of a spinal column segment, comprising:
a plate having at least a first hole therethrough between an upper surface and a lower surface of said plate to receive a bone fastener for passage into a first vertebra and at least a second hole therethrough between said upper surface and said lower surface of said plate to receive a bone fastener for passage into a second vertebra, wherein said lower surface of said plate is adapted for placement along an antero-lateral portion of the first and second vertebrae, and said first and second holes extend along first and second axes, respectively, said first and second hole axes being non-orthogonally oriented relative to said lower surface of said plate so that said bone fasteners positioned therethrough extend parallel to a sagittal plane of the spinal column segment when said plate is positioned along the antero-lateral portion of the first and second vertebrae.

20. The system of claim 19, wherein said bone fasteners extend into the vertebrae in a direction generally parallel to the sagittal plane when positioned through said plate holes.

21. The system of claim 19, wherein said plate includes spikes extending from said lower surface for embedding into the vertebrae.

22. The system of claim 19, wherein said plate includes a first thickness between said upper and lower surfaces along a lateral side thereof that is greater than a second thickness between said upper and lower surfaces along a medial side thereof.

23. The system of claim 22, wherein said lower surface of said plate is concavely curved between said medial and lateral sides.

24. The system of claim 23, wherein said upper surface of said plate is convexly curved between said medial and lateral sides.

25. The system of claim 23, wherein said upper surface of said plate includes a first portion lying in a first plane, a second portion lying in a second plane, and a riser portion therebetween, said first and second planes being parallel to one another.

26. The system of claim 25, wherein said plate includes:
a first hole in said first portion extending between said upper and lower surfaces orthogonally oriented to the first plane; and
a second hole in said second portion extending between said upper and lower surfaces orthogonally oriented to the second plane.

27. The system of claim 19, further comprising:
a second plate positionable antero-laterally along the first and second vertebrae on a side of the sagittal plane opposite the other plate; and
a length adjustable coupling mechanism positionable through a disc space between the first and second vertebrae and engageable to each of the plates.

28. A plating system for stabilization of a spinal column segment, comprising:
a plate including at least a first hole therethrough between an upper surface and a lower surface of said plate to receive a bone fastener for passage into a first vertebra and at least a second hole therethrough between said upper surface and said lower surface of said plate to receive a bone fastener for passage into a second vertebra, said lower surface of said plate being adapted for placement along an antero-lateral portion of the first and second vertebrae, said first and second holes extending along first and second axes, respectively, said first and second axes being oriented non-orthogonally to the lower surface of the plate, wherein said upper and lower surfaces extend between a lateral side and a medial side of said plate, said lateral side defining a thickness between said upper and lower surfaces that is greater than a thickness of said plate at said medial side.

29. The system of claim 28, wherein said lower surface of said plate is concavely curved between said medial and lateral sides.

30. The system of claim 29, wherein said upper surface of said plate is convexly curved between said medial and lateral sides.

31. The system of claim 28, wherein said lower surface includes a discal portion positionable extradiscally between the first and second vertebrae including a convexly curved surface profile in the direction between the first and second vertebrae.

32. The system of claim 31, wherein said lower surface includes first and second concavely curved pockets adjacent respective ends of said discal portion, said first and second pockets being structured to receive a cortical rim of a respective one of the first and second vertebrae when said lower surface of said plate member is positioned thereagainst.

33. A plating system for stabilization of a spinal column segment, comprising:
a plate including at least a first hole therethrough between an upper surface and a lower surface of said plate to receive a bone fastener for passage into a first vertebrae and at least a second hole therethrough between said upper surface and said lower surface of said plate to receive a bone fastener for passage into a second vertebra, said lower surface of the plate being structured for placement along an antero-lateral portion of the first and second vertebrae, said first and second holes extend along first and second axes, respectively, said first and second axes being oriented non-orthogonally to said lower surface of said plate, and said upper surface including a stair-stepped configuration.

34. The system of claim 33, wherein said upper surface of said plate includes a first portion lying in a first plane, a second portion lying in a second plane, and a riser portion therebetween, said first and second planes being parallel to one another.

35. The system of claim 34, wherein:
at least one of said first and second holes lies in said first portion and extends between said upper and lower surfaces and is orthogonally oriented to the first plane; and
at least one of said first and second holes lies in said second portion and extends between said upper and lower surfaces and is orthogonally oriented to the second plane.

36. The system of claim 35, wherein said lower surface is concavely curved between a medial side and a lateral side of said plate.

37. The system of claim 36, wherein said plate defines a thickness between said upper and lower surfaces, said thickness being greater adjacent said lateral side than said medial side.

38. An antero-lateral spinal plating system, comprising:
a first plate positionable antero-laterally along at least two vertebrae on a first side of a sagittal plane of a spinal column of a patient;
a second plate positionable antero-laterally along the at least two vertebrae on a second side of the sagittal plane opposite the first plate; and
a length adjustable coupling mechanism extending between the first and second plates and positionable in a disc space between the at least two vertebrae when said first and second plates are positioned antero-laterally along the at least two vertebrae, wherein said coupling mechanism is adjustable in length between the first and second plates.

39. The system of claim 38, wherein said first and second plates each include holes extending between upper and lower surfaces thereof for receiving bone fasteners.

40. The system of claim 38, wherein said first and second plates each include a lower surface convexly curved between a medial side and a lateral side of said respective plate.

41. The system of claim 40, wherein each of said plates further comprise spikes extending from said lower surface for engaging vertebrae of the spinal column segment.

42. An antero-lateral plate for a spinal column segment, comprising:
a body including an upper surface and a lower surface positionable antero-laterally along at least two vertebrae, said upper surface including a stair-stepped configuration having a first portion adjacent a medial edge of the plate and a second portion adjacent a lateral edge of the plate, said first and second portions being parallel to one another and each including at least one hole extending orthogonally therethrough for receiving a bone fastener to secure the plate to the at least two vertebrae.

43. The plate of claim 42, wherein said lower surface is convexly curved between said medial edge and said lateral edge.

44. The plate of claim 42, wherein said body defines a first thickness between said upper and lower surfaces adjacent said medial edge and a second thickness between said upper and lower surfaces adjacent said lateral edge, said second thickness being greater than said first thickness.
